(19) United States
(12) Patent Application Publication
(10) Pub. No.: US 2008/0027223 A1
(43) Pub. Date: Jan. 31, 2008

(54) POLYMORPHS OF ESZOPICLONE MALATE

(75) Inventors: Alex Mainfeld, Kfar-Saba (IL); Shlomit Wizel, Petah Tiqva (IL); Marioara Mendelovici, Rehovot (IL); Anita Liberman, Tel Aviv (IL); Tamas Koltai, Netanya (IL)

Correspondence Address: KENYON & KENYON LLP, ONE BROADWAY, NEW YORK, NY, 10004, US

(73) Assignee: TEVA PHARMACEUTICAL INDUSTRIES LTD., Petah Tiqva (IL)

(21) Appl. No.: 11/690,580

(22) Filed: Mar. 23, 2007

Related U.S. Application Data
(60) Provisional application No. 60/785,959, filed on Mar. 23, 2006.
(60) Provisional application No. 60/888,866, filed on Feb. 8, 2007.

(51) Int. Cl. C07D 487/04

(52) U.S. Cl. 544/350

(57) ABSTRACT

The present invention provides crystalline Eszopiclone malate form II, crystalline Eszopiclone form V, processes from preparing the crystalline Eszopiclone malate form II or V, pharmaceutical compositions comprising the crystalline Eszopiclone malate form II or V and methods of treating insomnia comprising administering the crystalline Eszopiclone malate form II or V.

### CROSS REFERENCE TO RELATED APPLICATIONS

The present application claims the benefits of U.S. Provisional Application Nos. 60/785,959 filed Mar. 23, 2006 and 60/888,866 filed Feb. 8, 2007, the disclosures of which are incorporated by reference herein.

### FIELD OF THE INVENTION

The present invention is related to crystalline form II and crystalline form V of Eszopiclone malate, (+)-6-(5-chloro-2-pyridinyl)-7(S)-(4-methylpiperazin-1-yl-carbonyloxy)-6,7-dihydro-5H-pyrrolo[3,4-b]pyrazine-5-one malic acid salt, methods for preparing the crystalline form II of Eszopiclone malate, crystalline form V of Eszopiclone malate and mixtures thereof, and pharmaceutical compositions comprising the crystalline form II and/or crystalline form V of Eszopiclone malate.

### BACKGROUND

Zopiclone, a non-benzodiazepine sedative-hypnotic useful for treating insomnia, is a racemate having a chemical name of 4-methyl-1-piperazinecarboxylic acid 6-(5-chloro-2-pyridinyl)-6,7-dihydro-7-oxo-5H-pyrrolo[3,4-b]pyrazin-5-yl ester, (±)-6-(5-chloro-2-pyridinyl)-6,7-dihydro-7-oxo-5H-pyrrolo[3,4-b]pyrazin-5-yl-4-methylpiperazine-1-carboxylate, or 6-(5-chloropyrid-2-yl)-5-(4-methylpiperazin-1-yl)carbonyloxy-7-oxo-6,7-dihydro-5H-pyrrolo[3,4-b]pyrazine, represented by formula I below.

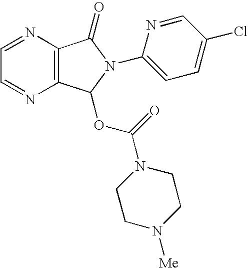

Eszopiclone is the S-enantiomer of zopiclone and is more active and less toxic than the racemic zopliclone according to U.S. Pat. No. 6,444,673 B 1. This drug has been marketed in the United States by Sepracor™ under the name Lunesta®, formerly known as Estorra®, having a CAS Registry Number of 138729-47-2. Eszopiclone has a chemical name of (+)-6-(5-chloro-2-pyridinyl)-7(S)-(4-methylpiperazin-1-yl-carbonyloxy)-6,7-dihydro-5H-pyrrolo[3,4-b]pyrazine-5-one and is represented with formula II below.

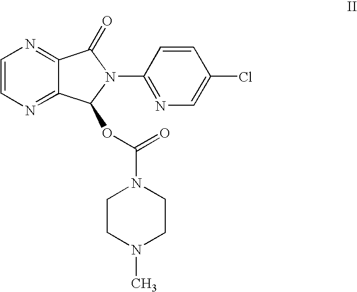

Eszopiclone can be prepared by optical resolution of racemic zopiclone. Blaschke, G. et al., Chirality, 1993, 5:419-421 discloses a method whereby ezsopiclone is prepared by recrystallization of zopiclone from methanol and acetone (4:6 volume ratio) in the presence of 0.5 equivalent of D-(+)-malic acid via the D-(+)-malic acid addition salt followed by crystallization from a 1:1 mixture of methanol/acetone. This reference discloses conversion of the eszopiclone D-malic salt to eszopiclone. The XRD for this sample was obtained and the crystalline form has been designated Form I (see IPCOM000134789D). U.S. Pat. No. 6,339,086 discloses an alternative method for the preparation of the Eszopiclone malate salt using one equivalent of D-(+)-Malic acid and racemic Zopiclone in a mixture of methanol/acetone in a 1:1.85 volume ratio. The product is then dried at 30-40° C. and 28 mm Hg. The wet product is Eszopiclone malate crystalline Form I while the dry product is designated crystalline Form IV (see IPCOM000134789D). US '086 also discloses conversion of the eszopiclone D-malic salt to eszopiclone.

The present invention relates to the solid state physical properties of Eszopiclone malate. These properties can be influenced by controlling the conditions under which eszopiclone malate is obtained in solid form. Solid state physical properties include, for example, the flowability of the milled solid. Flowability affects the ease with which the material is handled during processing into a pharmaceutical product. Another solid state physical property is the rate of dissolution in aqueous fluid or its behavior on compaction and its storage stability. Thus, there is a need in the art for additional crystalline forms of Eszopiclone malate.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others. A particular polymorphic form may also give rise to distinct spectroscopic properties that may be detectable by powder X-ray crystallography, solid state ¹³C-NMR spectrometry and infrared spectrometry.

The present invention relates to the solid state physical properties of Eszopiclone malate. These properties can be influenced by controlling the conditions under which Eszopiclone malate is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch, or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others. A particular polymorphic form may also give rise to distinct spectroscopic properties that may be detectable by powder X-ray crystallography, solid state ¹³C-NMR spectrometry, and infrared spectrometry. There is a need in the art for additional crystalline forms of Eszopiclone malate with improved solid state physical properties than previously known forms of Eszopiclone malate.

The present invention also relates to solvates of Eszopiclone malate. When a substance crystallizes out of solution, it may trap molecules of the solvent at regular intervals in the crystal lattice. Solvation also affects utilitarian physical properties of the solid state like flowability and dissolution rate.

One of the most important physical properties of a pharmaceutical compound, which can form polymorphs or solvates, is its solubility in aqueous solution, particularly the solubility in gastric juices of a patient. Other important properties relate to the ease of processing the form into pharmaceutical dosages, as the tendency of a powdered or granulated form to flow and the surface properties that determine whether crystals of the form will adhere to each other when compacted into a tablet.

The discovery of new polymorphic forms and solvates of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

### SUMMARY OF THE INVENTION

In one of the embodiments, the present invention is directed to crystalline Eszopiclone malate form II, characterized by data selected from: an x-ray powder diffraction (XRPD) pattern with peaks at about 11.6, 12.8, 18.1, 21.7, and 25.8±0.2 degrees 2θ; a DSC thermogram having peaks substantially as shown in FIG. 2; and both the XRPD pattern and DSC thermogram having peaks substantially as shown in FIG. 2.

Crystalline Eszopiclone malate form II can be characterized by data selected from: an x-ray powder diffraction (XRPD) pattern with peaks at about 11.6, 12.8, 18.1, 21.7, and 25.8±0.2 degrees 2θ; a DSC thermogram; and both the XRPD pattern and DSC thermogram; wherein the DSC thermogram has endotherms peaking at about 98° C., about 132° C., about 170° C. and about 196° C., or the DSC thermogram has endotherm A with a lower limit at about 89° C. and an upper limit at about 103° C., endotherm B with a lower limit at about 105° C. and an upper limit at about 139° C., endotherm C with a lower limit at about 163° C. and an upper limit at about 174° C., and endotherm D with a lower limit at about 174° C. and an upper limit at about 206° C.

In another embodiment of the present invention, a process is presented for preparing crystalline Eszopiclone malate form II comprising combining Eszopiclone-D-malate salt and C₁-C₇ chlorinated hydrocarbon.

In another embodiment of the present invention, a process is presented for preparing a mixture of crystalline Eszopiclone malate form II and Eszopiclone malate form I comprising combining Eszopiclone-D-malate salt and a mixture of C₁-C₇ chlorinated hydrocarbon and C₄-C₈ ester.

In another embodiment of the present invention, a process is presented for preparing crystalline Eszopiclone malate form II comprising slurrying Eszopiclone-D-malate in nitrile.

Optionally, Eszopiclone malate crystalline form II obtained by the above processes can be further converted into Eszopiclone by known methods, such as that disclosed in U.S. Pat. No. 6,339,086.

The present invention is also directed to crystalline Eszopiclone malate form V, characterized by an X-ray powder diffraction having peaks at about 4.5, 12.5, 16.4 and 17.0° 2θ±0.2° 2θ.

The present invention is further directed to a process for preparing Eszopiclone malate crystalline form V comprising maintaining Eszopiclone malate form I under about 80% to about 100% relative humidity, at about room temperature for about one or more day.

### BRIEF DESCRIPTION OF THE DRAWINGS

FIG. 1 illustrates a characteristic X-ray powder diffraction pattern of crystalline Eszopiclone malate salt form II.

FIG. 2 illustrates a characteristic DSC curve of crystalline Eszopiclone malate salt form II.

FIG. 3 illustrates a characteristic TGA curve of crystalline Eszopiclone malate salt form II.

FIG. 4 illustrates a characteristic X-ray powder diffraction pattern of crystalline Eszopiclone malate salt form V.

FIG. 5 illustrates a characteristic X-ray powder diffraction pattern of Eszopiclone malate salt form I.

FIG. 6 illustrates a characteristic X-ray powder diffraction pattern of Eszopiclone malate salt form IV.

### DETAILED DESCRIPTION OF THE INVENTION

As used herein, the term “RT” means room temperature. “Room temperature” as used herein preferably means a temperature of about 18° C. to about 25° C., preferably about 20° C. to about 25° C., and more preferably about 20° C. to about 22° C.

As used herein, the terms “Eszopiclone malate form I” and “Eszopiclone malate form IV” refer to the crystalline form obtained by performing the crystallization processes described in Blaschke, G. et al., Chirality, 1993, 5:419-421.

As mention herein, the Eszopiclone-D-malate salt used as the starting material in the processes for preparing crystalline Eszopiclone form II or V of the present invention may be prepared by any method known in the art, such as the one described in U.S. Pat. No. 6,339,086, the disclosures of which are hereby incorporated by reference.

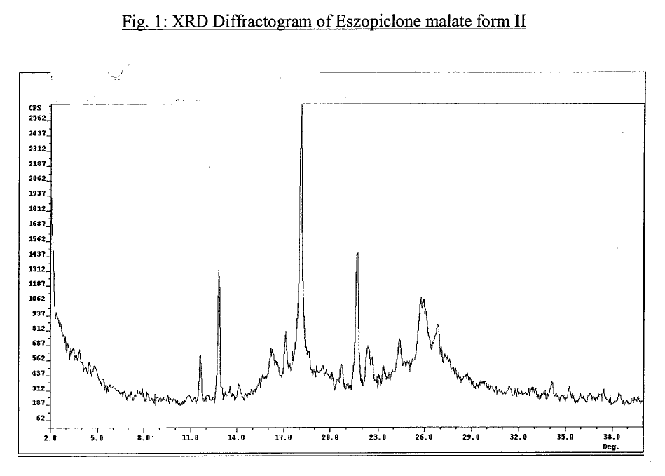

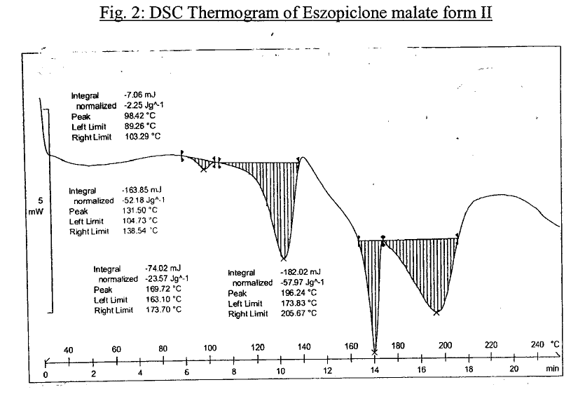

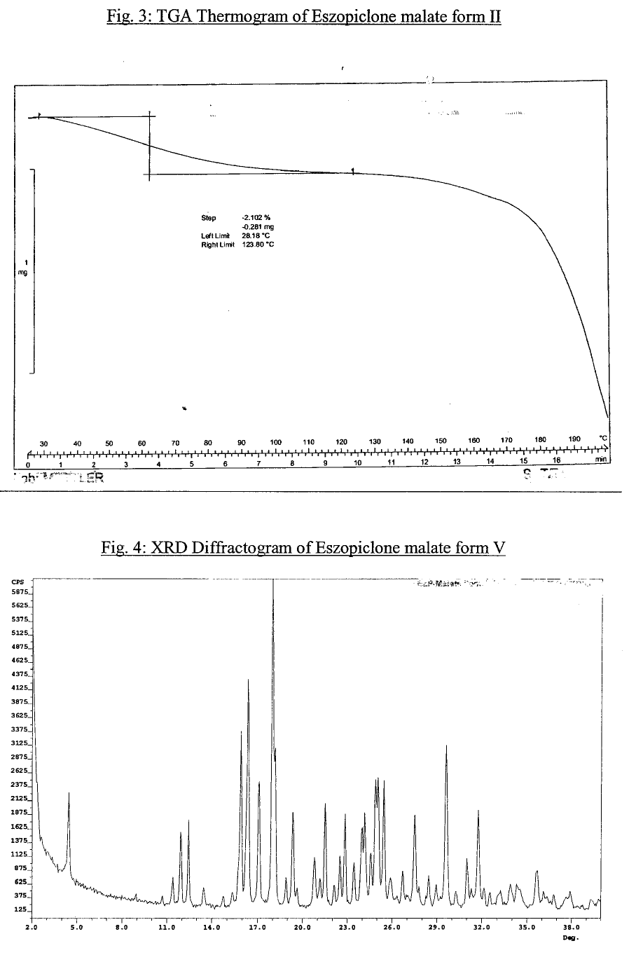

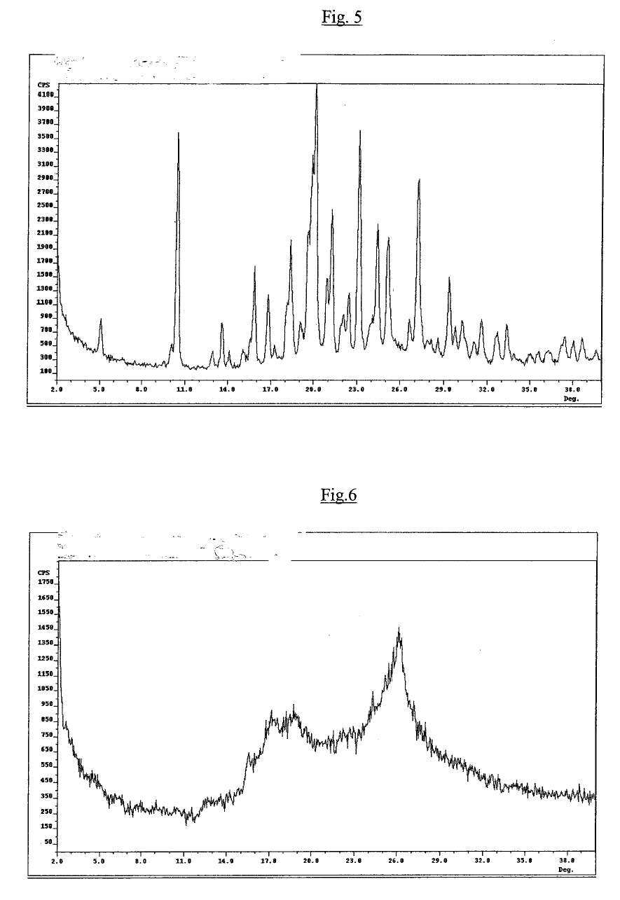

In one of the embodiments, the present invention is directed to a crystalline Eszopiclone malate form II, characterized by data selected from: an x-ray powder diffraction (XRPD) pattern with peaks at about 11.6, 12.8, 18.1, 21.7, and 25.8±0.2 degrees 2θ; a DSC thermogram having peaks substantially as shown in FIG. 2; and both the XRPD pattern and DSC thermogram. Eszopiclone malate form II may be further characterized by data selected from: an x-ray powder diffraction (XRPD) pattern with peaks at about: 16.2, 22.4, 24.4, and 26.9±0.2 degrees 2θ. The XRPD pattern, preferably, is as substantially depicted in FIG. 1. Preferably, the DSC thermogram has endotherms peaking at about 98° C., about 132° C., about 170° C. and about 196° C. or the DSC thermogram has endotherm A with a lower limit at about 89° C. and an upper limit at about 103° C., endotherm B with a lower limit at about 105° C. and an upper limit at about 139° C., endotherm C with a lower limit at about 163° C. and an upper limit at about 174° C., and endotherm D with a lower limit at about 174° C. and an upper limit at about 206° C. More preferably, endotherm A peaks at about 98° C., endotherm B peaks at about 132° C., endotherm C peaks about 170° C. and endotherm D peaks about 196° C.

Form II of Eszopiclone malate can be further characterized by a TGA thermogram showing a weight loss of about 2% in a temperature range of about 25° C. to about 120° C. that corresponds to the Karl Fischer titration. Form II of Eszopiclone malate may be in hydrate form.

In one embodiment of the present invention, a process is presented for preparing crystalline Eszopiclone malate form II comprising combining Eszopiclone-D-malate salt and C₁-C₇ chlorinated hydrocarbon.

The C₁-C₇ chlorinated hydrocarbon can be aromatic or, preferably, non-aromatic. Most preferably, the C₁-C₇ chlorinated hydrocarbon is methylene chloride.

Preferably, the process comprises: combining the Eszopiclone-D-malate salt and C₁-C₇ chlorinated hydrocarbon; heating; cooling; and maintaining.

Preferably, the weight (in gram) to volume (in ml) ratio of Eszopiclone-D-malate salt to C₁-C₇ chlorinated hydrocarbon is about 1:10 to about 1:30, most preferably about 1:20.

Preferably, the heating is to a temperature ranging from about room temperature to about reflux temperature, more preferably ranging from about room temperature to about 50° C., and most preferably ranging from about 30° C. to about 45° C. Preferably, stirring is performed during the heating step.

Preferably, after heating, a liquid phase and an oily phase are obtained. The liquid phase contains a chlorinated hydrocarbon solution of the malate. The oily phase contains the malate which is not dissolved.

Preferably, prior to the cooling step, the liquid phase and an oily phase are separated.

Preferably, the liquid phase is concentrated. Preferably, the liquid phase is concentrated by removal of a portion of the C₁-C₇ chlorinated hydrocarbon. Concentration of the liquid phase may be performed by any method known in the art, such as evaporation. The evaporation process may be a slow evaporation process. The evaporation may also be performed under vacuum. Optionally, after the concentration a reaction mixture is obtained.

Preferably, the cooling is to a temperature ranging from about 10° C. to about room temperature. Preferably, the cooling is performed on the liquid phase.

Preferably, the maintaining step is conducted at a temperature ranging from about 10° C. to about room temperature, more preferably, at about room temperature. Preferably, the maintaining step is conducted for about 0.5 to about 36 hours, more preferably for about 8 hours to about 36 hours. Preferably, the maintaining step is performed on the liquid phase

Optionally, during the maintaining stage, the mixture of the Eszopiclone-D-malate salt and C₁-C₇ chlorinated hydrocarbon is stirred. Preferably, the stirring is performed at a temperature ranging from about 10° C. to about room temperature, more preferably, at about room temperature. Preferably, the stirring is performed for about 16 hours.

Preferably, the obtained Eszopiclone malate crystalline form II is recovered.

In one embodiment of the present invention, a process is presented for preparing a mixture of crystalline Eszopiclone malate form II and Eszopiclone malate form I comprising combining Eszopiclone-D-malate salt, C₁-C₇ chlorinated hydrocarbon and C₄-C₈ ester.

Preferably, the C₄-C₈ ester is ethyl acetate.

The C₁-C₇ chlorinated hydrocarbon is aromatic or, preferably, non-aromatic. Most preferably, the C₁-C₇ chlorinated hydrocarbon is methylene chloride.

Preferably, when the C₁-C₇ chlorinated hydrocarbon is methylene chloride and the C₄-C₈ ester is ethyl acetate, the weight (in gram) to volume (in ml) ratio of Eszopiclone-D-malate salt to methylene chloride and ethyl acetate combined is about 1:20 to about 1:50, most preferably about 1:40. Preferably, the volume of methylene chloride and ethyl acetate are equal.

Preferably, the process comprises combining Eszopiclone-D-malate salt, C₁-C₇ chlorinated hydrocarbon and C₄-C₈ ester; heating; cooling; and maintaining.

Preferably, the heating is to a temperature ranging from about room temperature to about reflux temperature, more preferably ranging from about 20° C. to about 50° C., and most preferably ranging from about 40° C. to about 45° C. Preferably, stirring is performed during the heating step.

Preferably, after heating a slurry is obtained. Optionally, the slurry comprises a sticky solid.

Preferably, stirring is performed during the cooling step. Preferably, the cooling is to a temperature ranging from about 10° C. to about room temperature. More preferably, the cooling is to about room temperature.

Preferably, the maintaining step is conducted at a temperature ranging from about 10° C. to about room temperature, more preferably, at about room temperature. Preferably, the maintaining step is performed for about 0.5 to about 36 hours, more preferably for about 8 hours to about 36 hours. Preferably, stirring is performed during the maintaining step.

Preferably, the obtained mixture of Eszopiclone malate crystalline form II and Eszopiclone malate crystalline form I is further recovered.

In another embodiment of the present invention, a process is presented for preparing crystalline Eszopiclone malate form II comprising slurrying Eszopiclone-D-malate in nitrile.

Preferably, the weight (in gram) to volume (in ml) ratio of Eszopiclone-D-malate salt to nitrile is about 1:10 to about 1:30, most preferably about 1:20.

Preferably, the nitrile is C₂-C₄ nitrile. Preferably, the C₂-C₄ nitrile is acetonitrile.

Preferably, the process comprises: combining the Eszopiclone-D-malate and nitrile; heating; and cooling.

Preferably, the heating is to a temperature ranging from about room temperature to about reflux temperature, more preferably, at a temperature ranging from about 40° C. to about 60° C. Preferably, stirring is performed during the heating step.

Preferably, the cooling is performed at a temperature ranging from about 10° C. to about room temperature. Preferably, stirring is performed during the cooling step. Preferably, the cooling is performed for about 30 minutes to about four hours, preferably about three hours.

Preferably, the obtained Eszopiclone malate crystalline form II is further recovered.

Recovering Eszopiclone malate salt form II, obtained in the above processes, can be performed by any method, such as filtration, decantation and centrifugation, known in the art. Preferably the recovering comprises filtering, washing, and drying the solid. Washing is usually done with the same solvent used in the reaction. Preferably, the drying is by heating (such as in a vacuum oven) at about 25° C. to about 65° C., more preferably at about 35° C. Preferably, the drying is conducted under vacuum. Optionally, the drying of Eszopiclone malate crystalline form II is conducted for about 1 hour to about 20 hours, preferably about 4 hours to about 18 hours, and more preferably about 16 hours, to obtain dry crystalline Eszopiclone malate form II.

Optionally, Eszopiclone malate crystalline form II obtained by the above processes can be further converted into Eszopiclone by any of the known methods, such as that disclosed in U.S. Pat. No. 6,339,086 or U.S. Application No. 60/898,405 filed Jan. 31, 2007.

The present invention is also directed to crystalline Eszopiclone malate form V, characterized by an X-ray powder diffraction having peaks at about 4.5, 12.5, 16.4 and 17.0° 2θ±0.2° 2θ. The crystalline form V may be further characterized by X-ray powder diffraction peaks at about 11.4, 11.9, 18.0 and 21.5° 2θ±0.2° 2θ. The crystalline form V may be characterized by the XRPD pattern substantially as depicted in FIG. 4. Form V of Eszopiclone malate can be further characterized by a Karl Fischer titration showing a water content of about 5.2%. Form V of Eszopiclone malate may be in hydrate form.

The present invention is further directed to a process for preparing crystalline Eszopiclone malate form V comprising maintaining Eszopiclone malate form I at about 80% to about 100% relative humidity and about room temperature for about one or more days.

Preferably, the relative humidity is of about 100%.

Preferably, the maintaining is for about 3 days to about 7 days, more preferably, for about 6 days to about 7 days, most preferably, for about 7 days.

Preferably, the process for preparing crystalline Eszopiclone malate form V comprises maintaining Eszopiclone malate form I at about 100% RH and about room temperature for about 7 days.

Optionally, the crystalline Eszopiclone malate form V obtained by this process can be converted into Eszopiclone by any of the known methods, such as that disclosed in U.S. Pat. No. 6,339,086 or U.S. Application No. 60/898,405.

Conversion of Eszopiclone malate crystalline form II or form V into Eszopiclone can be by any means known in the art for conversion of Eszopiclone malate into Eszopiclone as for example the methods disclosed in Blaschke, G. et al., Chirality, 1993, 5:419-421,-U.S. Pat. No. 6,339,086 and U.S. Application No. 60/898,405, the disclosures of all of which are incorporated by reference.

Having described the invention with reference to certain preferred embodiments, other embodiments will become apparent to one skilled in the art from consideration of the specification. The invention is further demonstrated by reference to the following examples describing in detail the preparation of the composition and methods of use of the invention. It will be apparent to those skilled in the art that many modifications, both to materials and methods, may be practiced without departing from the scope of the invention.

### EXAMPLES

### Physical Experimental Methodology Used for Analyzing Forms II and V

XRPD measurements were performed using an Scintag X-Ray powder diffractometer model X'TRA, Cu-tube, solid state detector, with a Peltier detector and a round standard aluminum sample holder with round zero background quartz plate was used. The scanning parameters were as follows: the range was 2° to 40° 2θ with a continuous scan at a rate of 3° per minute. The accuracy of peak positions can be defined as ±0.2° 2θ due to experimental differences, such as instrumentations, sample preparations, and the like.

Differential scanning calorimetry (DSC) measurements were performed on a Mettler Toledo DSC 821e/500 with a sample having a weight of about 3 to about 5 mg. The heating rate was 10° C./minute in a crucible having 3 holes with a nitrogen stream flow rate of about 40 ml per minute over a scan range of from about 30° to about 215° C.

The thermal gravimetric analysis (TGA) was performed on a Mettler TG50 instrument, with a sample having a weight of about 7 to about 15 mg, at a heating rate of about 10° C./minute, in a nitrogen gas stream having a flow rate of about 40 ml per minute, over a scan range of about 25° to about 200° C.

### Example 1

### Preparation of Eszopiclone Malate Form II

Eszopiclone-D-malate salt (1 gr, 93.5% EZP) and methylene chloride (20 ml) were stirred magnetically at 40° C. No complete dissolution was observed and the solid became oily. The stirring at 40° C. for 1 h did not improve the dissolution and the two phases separate from the reaction mixture: an oily solid and the solvent. The solvent was concentrated. The stirring at 40° C. for 1 h did not improve the dissolution and the two phases separate from the reaction mixture: an oily solid and the solvent. The solvent was concentrated and the stirring was continued overnight at the room temperature. The solid became crystalline during the stirring and the mixture was diluted with additional volume of CH₂Cl₂ (2 ml). The solid was filtered and dried in vacuum oven at 35° C. to give 0.1 gr. solid (10% yield, purity 99.13% Eszopiclone malate form II based on XRD analysis).

### Example 2

### Preparation of Eszopiclone Malate Form II

Eszopiclone-D-malate salt (1 g) was slurried in acetonitrile (20 ml) at 60° C. for 1 hour. After this the slurry was cooled and stirred during three hours at about 20° C., the solid was filtrated, washed with acetonitrile (5 ml) and dried at about 35° C. for about 16 hours. The dried solid was Eszopiclone malate form II (yield: 84%).

### Example 3

### Preparation of a Mixture Eszopiclone Malate Form II and Form I

Eszopiclone-D-Malate salt (1 gr) was stirred magnetically with a mixture of 20 ml CH₂Cl₂ and 20 ml EtOAc (40 ml total), at a temperature of about 45° C. No dissolution was observed and the solid became sticky. The heating was stopped and the slurry was cooled to room temperature and stirred for 3 h. The solid was filtered, washed with a mixture of 1:1 CH₂Cl₂/EtOAc (10 ml) and dried in a vacuum oven at 35° C. The solid was analyzed by XRD and identified as a mixture of Eszopiclone malate form I and form II. (Yield 47%).

### Example 4

### Preparation of Eszopiclone Malate Form V

200 mg of Eszopiclone malate form I was placed in a container and stored for 7 days under 100% RH at room temperature. After storage, the sample was analyzed by XRD and found to be Eszopiclone malate form V.

1. Crystalline Eszopiclone malate form II, characterized by data selected from: an x-ray powder diffraction (XRPD) pattern with peaks at about 11.6, 12.8, 18.1, 21.7, and 25.8±0.2 degrees 2θ; a DSC thermogram having peaks substantially as shown in FIG. 2; and both the XRPD pattern and the DSC thermogram.

2. The Eszopiclone malate form II of claim 1, wherein the XRPD pattern has further characterizing peaks at about 16.2, 22.4, 24.4, and 26.9±0.2 degrees 2θ.

3. The Eszopiclone malate form II of claim 2 having a XRPD pattern substantially as depicted in FIG. 1.

4. The Eszopiclone malate form II of claim 2, wherein the DSC thermogram has endotherms peaking at about 98° C., about 132° C., about 170° C. and about 196° C., or the DSC thermogram has endotherm A with a lower limit at about 89° C. and an upper limit at about 103° C., endotherm B with a lower limit at about 105° C. and an upper limit at about 139° C., endotherm C with a lower limit at about 163° C. and an upper limit at about 174° C., and endotherm D with a lower limit at about 174° C. and an upper limit at about 206° C.

5. The Eszopiclone malate form II of claim 2, having a TGA thermogram showing a weight loss of about 2% in a temperature range of about 25° C. to about 120° C.

6. A process for preparing the Eszopiclone malate form II of claim 1, comprising combining Eszopiclone-D-malate salt and C₁-C₇ chlorinated hydrocarbon to form the Eszopiclone malate form II.

7. The process of claim 6, wherein the C₁-C₇ chlorinated hydrocarbon is non-aromatic.

8. The process of claim 7 wherein the C₁-C₇ chlorinated hydrocarbon is methylene chloride.

9. The process of claim 6, wherein the process comprises combining the Eszopiclone-D-malate salt and C₁-C₇ chlorinated hydrocarbon to form a mixture; heating the mixture; cooling the heated mixture; and maintaining the cooled mixture to obtain the Eszopiclone malate form II.

10. The process of claim 6, wherein the weight (in gram) to volume (in ml) ratio of the Eszopiclone-D-malate salt to the C₁-C₇ chlorinated hydrocarbon is about 1:10 to about 1:30.

11. The process of claim 10, wherein the weight (in gram) to volume (in ml) ratio of the Eszopiclone-D-malate salt to the C₁-C₇ chlorinated hydrocarbon is about 1:20.

12. The process of claim 9, wherein the heating is to a temperature ranging from about room temperature to about reflux temperature.

13. The process of claim 12, wherein the heating is to a temperature ranging from about 30° C. to about 45° C.

14. The process of claim 9, wherein the mixture is stirred during the heating step.

15. The process of claim 9, wherein after heating, a liquid phase and an oily phase are obtained.

16. The process of claim 15 further comprising separating the liquid phase and oily phase.

17. The process of claim 16, wherein the liquid phase is concentrated.

18. The process of claim 17, wherein the liquid phase is concentrated by removal of a portion of the C₁-C₇ chlorinated hydrocarbon.

19. The process of claim 9, wherein the cooling is to a temperature ranging from about 10° C. to about room temperature.

20. The process of claim 19, wherein the cooling is performed on the liquid phase.

21. The process of claim 9, wherein the mixture is stirred during the cooling step.

22. The process of claim 9, wherein the maintaining step is conducted at a temperature ranging from about 10° C. to about room temperature.

23. The process of claim 9, wherein the maintaining step is conducted for about 0.5 to about 36 hours.

24. The process of claim 9, wherein the mixture is stirred during the maintaining step.

25. The process of claim 6, further comprising recovering the formed crystalline Eszopiclone malate form II.

26. A process for preparing a mixture of the crystalline Eszopiclone malate form II of claim 1 and Eszopiclone malate form I comprising combining Eszopiclone-D-malate salt, C₁-C₇ chlorinated hydrocarbon and C₄-C₈ ester to form the mixture of crystalline Eszopiclone malate form II and Eszopiclone malate form I.

27. The process of claim 26, wherein the C₁-C₇ chlorinated hydrocarbon is non-aromatic.

28. The process of claim 27, wherein the C₁-C₇ chlorinated hydrocarbon is methylene chloride.

29. The process of claim 26, wherein the C₁-C₇ chlorinated hydrocarbon is methylene chloride and the C₄-C₈ ester is ethyl acetate.

30. The process of claim 29, wherein the weight (in gram) to volume (in ml) ratio of the Eszopiclone-D-malate salt to the methylene chloride and ethyl acetate combined is about 1:20 to about 1:50.

31. The process of claim 26, wherein the process comprises combining the Eszopiclone-D-malate salt, C₁-C₇ chlorinated hydrocarbon and C₄-C₈ ester to form a combined mixture; heating the combined mixture; cooling the heated combined mixture; and maintaining the cooled combined mixture to obtain the mixture of crystalline Eszopiclone malate form II and Eszopiclone malate form I.

32. The process of claim 31, wherein the heating is to a temperature ranging from about room temperature to about reflux temperature.

33. The process of claim 32, wherein the heating is to a temperature ranging from about 20° C. to about 50° C.

34. The process of claim 31, wherein after heating a slurry is obtained.

35. The process of claim 34, wherein the slurry comprises a sticky solid.

36. The process of claim 31, wherein the cooling is to a temperature ranging from about 10° C. to about room temperature.

37. The process of claim 31, wherein the maintaining step is conducted at a temperature ranging from about 10° C. to about room temperature.

38. The process of claim 31, wherein the maintaining step is conducted for about 0.5 to about 36 hours.

39. The process of claim 31, wherein the combined mixture is stirred during the maintaining step.

40. The process of claim 26, further comprising recovering the formed mixture of crystalline Eszopiclone malate form II and Eszopiclone malate form I.

41. A process for preparing the crystalline Eszopiclone malate form II of claim 1, comprising slurrying Eszopiclone-D-malate in nitrile to form the crystalline Eszopiclone malate form II.

42. The process of claim 41, wherein the weight (in gram) to volume (in ml) ratio of the Eszopiclone-D-malate salt to the nitrile is about 1:10 to about 1:30.

43. The process of claim 41, wherein the nitrile is C₂-C₄ nitrile.

44. The process of claim 43, wherein the C₂-C₄ nitrile is acetonitrile.

45. The process of claim 41, wherein the process comprises combining the Eszopiclone-D-malate and nitrile to obtain a mixture; heating the mixture; and cooling the heated mixture to obtain the crystalline Eszopiclone malate form II.

46. The process of claim 45, wherein the heating is to a temperature ranging from about room temperature to about reflux temperature.

47. The process of claim 46, wherein the heating is to a temperature ranging from about 40° C. to about 60° C.

48. The process of claim 41, wherein the mixture is stirred during the heating step.

49. The process of claim 41, wherein the cooling is to a temperature ranging from about 10° C. to about room temperature.

50. The process of claim 41, wherein the mixture is stirred during the cooling step.

51. The process of claim 41, further comprising recovering the formed crystalline Eszopiclone malate form II.

52. Crystalline Eszopiclone malate form V, characterized by an X-ray powder diffraction (XRPD) pattern having peaks at about 4.5, 12.5, 16.4 and 17.0° 2θ±0.2° 2θ.

53. The crystalline Eszopiclone malate form V of claim 52, further characterized by the XRPD pattern having peaks at about 11.4, 11.9, 18.0 and 21.5° 2θ±0.2° 2θ.

54. The crystalline Eszopiclone malate form V of claim 52, wherein the XRPD pattern is substantially as depicted in FIG. 4.

55. The crystalline Eszopiclone malate form V of claim 52, further characterized by a water content of about 5.2% determined via Karl Fischer titration.

56. A process for preparing the crystalline Eszopiclone malate form V of claim 52, comprising maintaining Eszopiclone malate form I at a relative humidity of about 80% to about 100% and about room temperature for about one or more days.

57. The process of claim 56, wherein the relative humidity is of about 100%.

58. The process of claim 56, wherein the maintaining step is performed for about 3 days to about 7 days.

59. The process of claim 56, wherein the process comprises maintaining the Eszopiclone malate form I at about 100% RH and about room temperature for about 7 days.

60. A process for preparing Eszopiclone comprising converting the crystalline Eszopiclone malate form II or crystalline Eszopiclone malate form V obtained by one of the processes of claims 6, 26, 41 and 56 to Eszopiclone.

61. Crystalline Eszopiclone malate form II characterized by data selected from: an x-ray powder diffraction (XRPD) pattern with peaks at about 11.6, 12.8, 18.1, 21.7, and 25.8±0.2 degrees 2θ; a DSC thermogram; and both the XRPD pattern and DSC thermogram; wherein the DSC thermogram has endotherms peaking at about 980C, about 132° C., about 170° C. and about 196° C., or the DSC thermogram has endotherm A with a lower limit at about 89° C. and an upper limit at about 103° C., endotherm B with a lower limit at about 105° C. and an upper limit at about 139° C., endotherm C with a lower limit at about 163° C. and an upper limit at about 174° C., and endotherm D with a lower limit at about 174° C. and an upper limit at about 206° C.

62. The Eszopiclone malate form II of claim 61, wherein endotherm A peaks at about 98° C., endotherm B peaks at about 132° C., endotherm C peaks about 170° C. and endotherm D peaks about 196° C.

63. The Eszopiclone malate form II of claim 61, wherein the XRPD pattern has further characterizing peaks at about 16.2, 22.4, 24.4, and 26.9±0.2 degrees 2θ.

64. The Eszopiclone malate form II of claim 63 having a XRPD pattern substantially as depicted in FIG. 1.

65. The Eszopiclone malate form II of claim 61, having a water content of about 2% determined via Karl Fischer titration.
